(19) United States
(12) Patent Application Publication
(10) Pub. No.: US 2021/0271060 A1
(43) Pub. Date: Sep. 2, 2021

(54) Systems and Methods for Improved Axial Resolution in Microscopy Using Photoswitching and Standing Wave Illumination Techniques

(71) Applicant: c/o THE UNITED STATES OF AMERICA, AS REPRSENTED BY THE SECRETARY, DEPARTMENT OF HEALTH AND HUMAN SER, Bethesda, MD (US)

(72) Inventors: Hari Shroff, Bethesda, MD (US); John Paul Giannini, Bethesda, MD (US); Yicong Wu, Bethesda, MD (US); Patrick Jean La Riviere, Bethesda, MD (US); Min Guo, Bethesda, MD (US); Jiji Chen, Bethesda, MD (US); Harshad Vishwasrao, Bethesda, MD (US); Xuesong Li, Rockville, MD (US)

(21) Appl. No.: 17/253,090

(22) PCT Filed: Jun. 27, 2019
(86) PCT No.: PCT/US2019/039551

§ 371 Date: Dec. 16, 2020

Related U.S. Application Data
(60) Provisional application No. 62/789,210, filed on Jan. 7, 2019.
(60) Provisional application No. 62/693,750, filed on Jul. 3, 2018.

(51) Int. Cl. G02B 21/00; G01N 21/64; G02B 21/36; G01N 33/58

(52) U.S. Cl. CPC G02B 21/0032; G01N 21/6428; G01N 21/6458; G02B 21/0076; G01N 2021/6478; G02B 21/0048; G01N 33/582; G01N 2021/6439; G02B 21/367

(57) ABSTRACT

Various embodiments for systems and methods for improved axial resolution in a microscopy using photoswitching and standing-wave illumination techniques are described.

### FIELD

The present disclosure generally relates to improving axial resolution in microscopy, and in particular to systems and methods for improved axial resolution in instant structured illumination microscopy using photoswitching and standing-wave illumination techniques.

### BACKGROUND

One known method of increasing the accessible axial spatial frequencies (and thus the resolution) in conventional, widefield fluorescence microscopy is to use standing-wave illumination. In this method, two counter-propagating coherent beams are superposed at the imaging focal plane. Interference between the beams results in sharp, periodic illumination fringes with periodicity given by λ/(2 n cos Θ) where λ is the wavelength of illumination, n the index of the media and Θ the ‘crossing angle’ of the beams, i.e. the angle relative to the vertical illustrated in FIG. 1A. As shown, two beams cross at common angle Θ with respect to a vertical axis (dashed line, which also represents the vertical optical axis). Referring to FIG. 1B, an example microscopy setup 10 is illustrated with an arrangement of an objective lens 12 and a mirror 14 that forms counter-propagating beams in which the resulting interference pattern show sharp dark/bright intensity fringes. For example, for θ=0, n=1.33, and λ=405 nm, the periodicity between fringes is 152 nm, and the spacing between dark/bright fringes is only ˜76 nm—implying that, in principle, structure on this length-scale can be observed. Such an interference pattern 16 can be set up by introducing mutually coherent light beams through opposed objectives (or introducing a single collimated beam through an objective and folding it back onto itself with a mirror, whereby a fringe pattern will be formed within the coherence length of the illumination). These (or conceptually similar) illumination patterns form the basis of standing-wave microscopy, 4pi microscopy, and super-resolution I5S microscopy.

In thin samples (thickness<λ), introducing a standing-wave illumination pattern as described above can yield valuable subdiffractive information. Moving the standing-wave pattern relative to the sample (i.e. altering the phase of the standing-wave pattern) causes alternating sample regions within the focal plane to glow, thereby allowing axial features finer than the axial spread of the point spread function to be discerned. However, for samples that are substantially thicker, three problems arise. First, out-of-focus fluorescence can swamp in-focus signal. Second, the repeating axial nature of the high frequency interference pattern implies an ambiguity about ‘which fringe is which’, i.e. fringes within the point spread function (PSF) create ringing artifacts in the reconstructed images (an alternative explanation of this problem is that the high frequencies of the illumination are aliased into the passband of the microscope). Third, there is an intermediate frequency ‘gap’ that exists in the reconstructed images because the frequency f of the standing wave lies outside the band limit of the microscope's optical transfer function (OTF).

Frequency gaps when using a standing wave illumination are shown in FIGS. 2A and 2B. Referring to FIG. 2A, an example of the XZ OTF (kz, vertical, kx, lateral) is shown when using instant SIM illumination. The axial diffraction limit is given by the boundary of the solid white ellipse. Referring to FIG. 2B, the OTF when using standing-wave illumination, e.g. by first photoactivating molecules with a standing wave having spatial frequency f and different phases (solving the aliasing problem), imaging the photoactivating molecules using an instant SIM system, and then deconvolving the resulting images. Now additional copies of the OTF exist, centered at +/−f in addition to the original OTF at the DC component (red dots). The axial spatial resolution is improved; however, substantial gaps at intermediate spatial frequencies existed—these gaps are not ‘covered’ by the detection OTF of the underlying microscope as f lies outside the band limit.

These latter two issues are not solved in traditional 4pi microscopy, but are addressed in a I5S system, which uses a complex three-beam interference pattern, interference of both excitation and emission light, and multiple images per focal plane to ‘fill in’ the missing axial spatial frequencies and reassign them to their proper location in frequency space.

However, the I5S system introduces the following problems. First, the I5S system has so far required two-objective interferometry and a complex beam setup which makes the system difficult to align and build due to the need to maintain the optics along two separate paths (one for each objective) aligned to a spatial precision much better than λ. Second, the I5S system requires fifteen images per focal plane to achieve improved axial resolution improvement which significantly slows down the imaging process and thus far limits imaging to fixed cells. Third, no confocal pinhole is employed by the I5S system such that in densely labeled specimens Poisson noise from out-of-focus light will limit contrast in the focal plane. Finally, the beam illumination scheme of the I5S system is highly specialized since the same illumination scheme is used both for creating the axial resolution improvement and the lateral resolution improvement. Because the resolution improvement is coupled, this method is not easily adapted to confocal geometries.

It is with these observations in mind, among others, that various aspects of the present disclosure were conceived and developed.

### BRIEF DESCRIPTION OF THE DRAWING

FIG. 1A is an illustration showing two beams crossing at a common angle with respect to a vertical axis and FIG. 1B is a simplified illustration showing an example microscopy setup with objective lens and mirror arrangement and the resulting interference pattern.

FIG. 2A is an image of an XZ optical transfer function when using instant SIM and FIG. 2B is an image of the XZ optical transfer function when using standing-wave illumination.

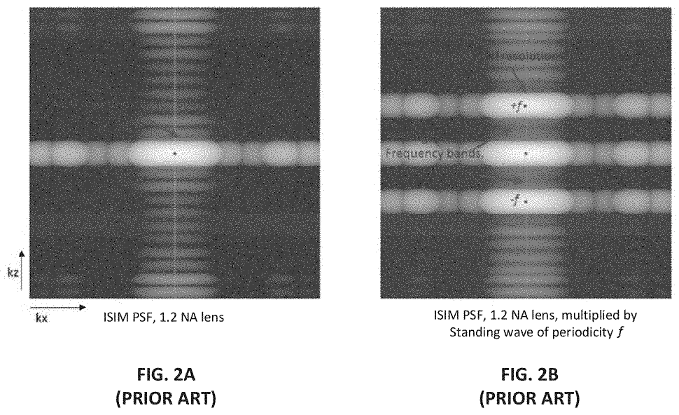

FIG. 3 is a simplified illustration showing a standing-wave microscopy system.

FIG. 4 is an illustration showing the use of multiple patterns for “filling in” intermediate spatial frequencies, each of the multiple patterns having a different periodicity.

FIG. 5 is a simplified block diagram showing an embodiment of a microscopy system.

FIGS. 6A-6E show simulated images produced by the microscopy system of FIG. 5 showing increased axial resolution.

FIG. 7 is a simplified block diagram showing an embodiment of a microscopy system having illuminator/reflector optics for illuminating with standing waves of different periodicity.

FIG. 8A are images of sharp sinusoidal illumination at different phases (left) in relation to an image of uniform illumination (right); FIG. 8B show the blue circle as representing the objective back focal plane and the red dots represent the illumination pattern at the back focal plane; FIG. 8C is an image of a sharper illumination pattern introduced at the sample.

FIG. 9 is a simplified illustration showing an embodiment of the standing-wave microscopy system for supplying an axial illumination structure of intermediate and finest periodicity for generating the standing wave.

Corresponding reference characters indicate corresponding elements among the view of the drawings. The headings used in the figures do not limit the scope of the claims.

### DETAILED DESCRIPTION

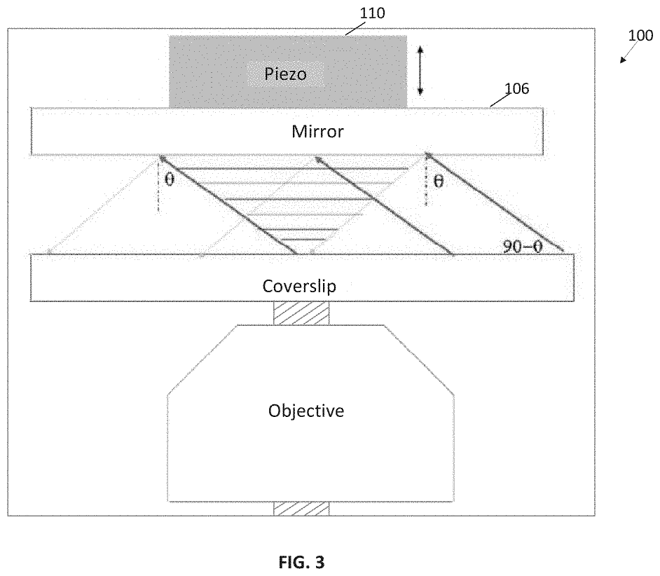

It is well known that the axial resolution of conventional widefield fluorescence microscopy is limited to a range between ˜500-700 nm. Systems and methods that can further improve axial resolution are of great interest in fluorescence microscopy, as such improvements would allow for greater detail to be observed in biological samples. Various embodiments related to systems and methods that enable axial resolution down to ˜100 nm by acquiring only four extra images at each focal plane for a total of five images per focal plane (instead of one image) are disclosed herein that address these deficiencies. Given the modest number of additional images required to improve axial resolution of images, embodiments of the present system and method and can be applied for sustained volumetric imaging (‘4D imaging’) in live cells, which is currently not possible with other microscopy techniques. Furthermore, the present system and method is flexible and can be combined with other super-resolution microscopes that allow further improvements in lateral resolution for those types of microscopy systems. In some embodiments, the microscopy system includes a spatial light modulator positioned conjugate to the sample being illuminated for activating the sample with a standing wave. In some embodiments, a method and related system is disclosed for supplying an axial illumination structure of intermediate and finest periodicity for the standing wave. In some embodiments, a triple beam-splitting device is used to generate three mutually coherent light beams from a single light beam that interferes at the sample to produce lower spatial frequency axial fringes necessary for achieving higher axial resolution. Referring to the drawings, embodiments of a microscopy system using photoswitching and standing wave illumination techniques are illustrated and generally indicated as 100, 200, 300 and 400 in FIGS. 3-9 are disclosed.

### Photoswitching Standing-Wave Illumination

The present system and method is directed to decoupling standing-wave illumination from fluorescence excitation and readout using a photoswitching technique, and utilizing a compact standing-wave reflector and illuminator arrangement. Together, these elements allow axial super-resolution at much higher speeds than previously possible. Although the present system and method can be applied to a large class of microscopes (e.g. spinning-disk confocal microscopes and widefield microscopes), the present disclosure describes, by way of example, the inventive concept being applied to instant structured illumination microscopy (iSIM⁴) since combining iSIM with photoswitching and standing-wave illumination techniques enables confocal, 3D super-resolution microscopy having ˜100 nm axial resolution and high frame rates consistent with live-cell imaging.

Using Photoswitching Technique to Decouple Standing Wave Illumination from Fluorescence Excitation and Readout

By using a reversibly switchable fluorescent molecule such as rsEGP2 and employing a standing-wave illuminator/reflector arrangement (described further below), fluorescence excitation and readout may be performed using a large variety of confocal (or other) microscope geometries (whose excitation optics and thus illumination remain virtually unchanged relative to the base microscope) as the axial resolution enhancement may be ‘added on’ to the underlying microscope. Additionally, by using an activation wavelength in addition to the typical fluorescence excitation wavelength, axial resolution is slightly improved since λ_{activation}<λ_{excitation}.

### Standing-Wave Illuminator/Reflector

Referring to FIG. 3, a first embodiment of a microscopy system for utilizing the photoswitching and standing wave illumination techniques, designated 100, transmits a collimated beam 102 through an objective 104 and uses a mirror 106 to reflect the collimated beam 102 back. The interference pattern 108 generated between the two collimated beams 102—the transmitted collimated beam 102A and the reflected collimated beam 102B results in a standing wave. In some embodiments, fine control over the phase of the standing-wave pattern is achieved by translating a piezoelectric device 110 affixed to the mirror 106.

As further shown, for example, an illumination beam 102A (dark blue rays) is transmitted through the objective lens 104 and coverslip 114 and mirror 106 positioned parallel to the coverslip 114 reflects the collimated beam 102B back (lighter rays). Interference between the two beams produces a standing wave pattern 116 (red lines) in the region of beam overlap. In some embodiments, piezoelectric device 110 affixed to the mirror 106 translates the mirror 106 that provides fine control of the phase of the standing-wave pattern 116.

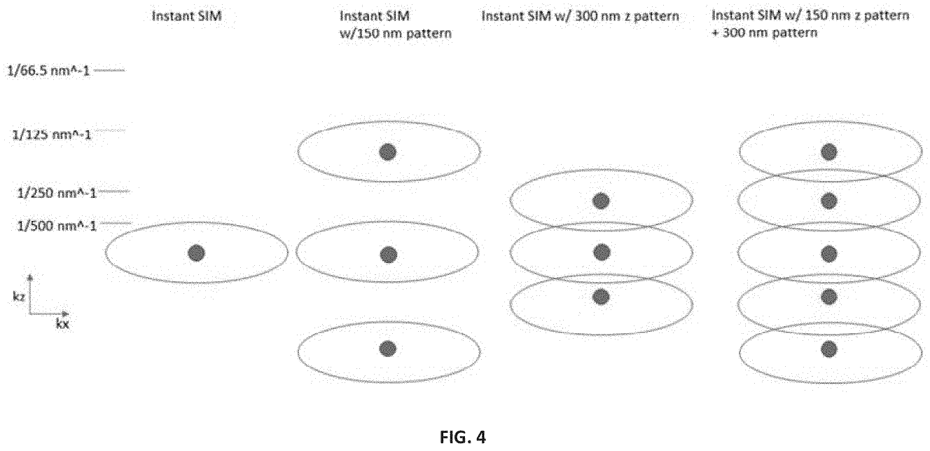

As discussed above, a single on-axis standing-wave pattern that produces a bright/dark fringe spacing substantially less than λ_{activation} (e.g. the 76-nm spacing mentioned above) enables higher resolution at the expense of a spatial frequency gap, which in turn leads to artifacts in the reconstructed image. However, if additional patterns with coarser spacings that lie in the intermediate frequency gap are used, the frequency gap can be ‘filled in’ as shown in FIG. 4, in which the present system and method “fills in” intermediate spatial frequencies by using multiple patterns, each of different periodicity. The leftmost column of FIG. 4 shows that in a conventional instant SIM system (not shown), the optical transfer function (OTF, ellipse) limits axial resolution to ˜500 nm. The middle left column of FIG. 4 shows that using a standing wave with 150 nm periodicity increases axial spatial frequencies, but also produces a gap at intermediate spatial frequencies, because the periodicity (blue dots) lies well outside the instant SIM cutoff. The middle right column of FIG. 4 shows that using a coarser standing wave pattern (e.g. 300 nm) produces an increased resolution without frequency gaps, since the OTF copies overlap in frequency space. However, the maximum resolution is less than using a finer pattern. The rightmost column of FIG. 4 shows that using both finer and coarser patterns results in the best axial resolution without missing spatial frequencies.

The question now is how to generate and apply additional patterns of the appropriate periodicity. According to the present system and method one simple way of altering the fringe spacing is to vary θ. For example, for 405 nm illumination (i.e. λ_{activation}), n=1.33, θ=0 degrees implies a periodicity of 152 nm (fringe spacing 76 nm) and θ=60 degrees implies a periodicity of 304 nm (fringe spacing 152 nm). In order to quickly vary θ at the sample plane, the following illumination setup as described below was conceived.

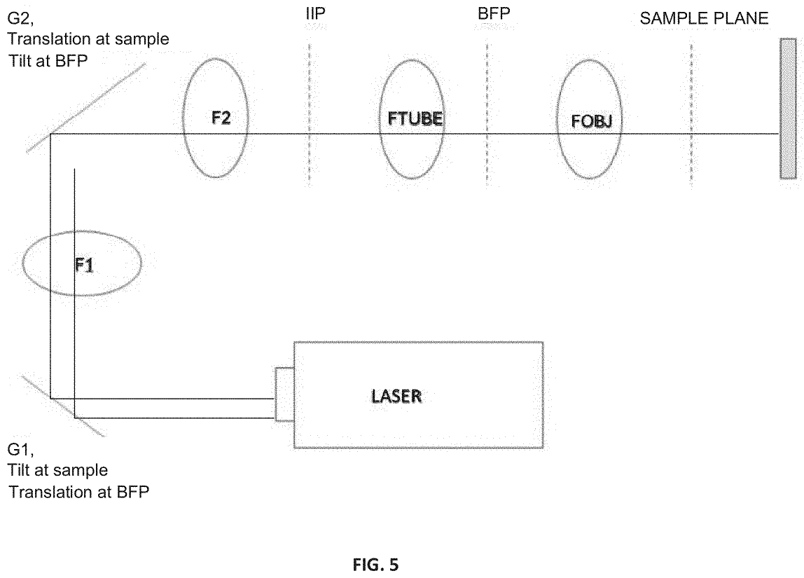

As shown in FIG. 5, a second embodiment of the microscopy system for utilizing the photoswitching and standing-wave illumination techniques, designated 200, includes an illumination source 202, such as a laser, for producing a laser beam 204 that is reflected off a first galvanometric mirror scanner (G1) 205 through a first lens 206 and reflects off a second galvanometric mirror scanner (G2) 207 before being relayed through a telescope composed of second lens 208 and third lens FTUBE 212 onto the back focal plane of objective lens (FOBJ) 216 before being finally focused onto the sample 218. A mirror 220 then reflects the illumination back onto the sample as in FIG. 4. In one arrangement, the first galvanometric mirror scanner (G1) 205 is positioned in a location conjugate to the sample 218, i.e. imaged first to intermediate image plane IIP 210 by a pair of lenses, first lens F1 206 and second lens F2 208 (in a 4f configuration) and then to the sample 218 by a pair of lenses, FTUBE 212 and FOBJ 216 (also in a 4f configuration). Importantly, scanning first galvanometric mirror (G1) 205 tilts the standing-wave pattern at the sample plane 218 (varying θ), thus changing the standing-wave periodicity. Intermediate lenses F2 208 and FTUBE 212 ensure that the second galvanometric mirror scanner (G2) 207 is conjugate to the back focal plane of the objective (BFP) 214, thus tilting the collimated beam at the BFP 214 or translating it at the sample 218 and ensuring that the standing wave stays centered on the sample 218. A dichroic mirror (not shown) positioned in the vicinity of IIP 210 couples in/out the instant SIM path, e.g. for providing excitation illumination of a different wavelength and spatial patterning and to direct fluorescence from the sample to an imaging system (not shown).

In some embodiments, the first and second galvanometric scanners (G1) 205 and (G2) 207 provide independent control of the position and angle of the collimated light 204 at the back focal plane 214, and thus change angle or position, respectively, in the sample plane. By varying the angle of the first galvanometric mirror scanner (G1) 205 appropriately, patterns of periodicity ranging from λ_{activation}/2n to λ_{activation}/(2n cos θ_{MAX}) can be created by the microscopy system 200 at the sample plane 218, where θ_{MAX} is the maximum half angle allowed by the objective lens (e.g. 64.5 degrees for a 60×, 1.2 NA water lens). By varying the angle of the second galvanometric mirror scanner (G2) 207 appropriately, the patterns may be translated at the sample plane 218, ensuring that these patterns illuminate the sample 220.

An additional advantage of this ‘single objective’ setup of the microscopy system 200 with a mirrored reflector is that the alignment of the microscopy system 200 is likely far more stable and resistant to mechanical/thermal drift than a classic 2-objective setup (as is used e.g. in I5S or 4pi microscopy systems): since a common optical path is employed for both direct and reflected beams only the sample-to-mirror distance must be kept stable to within λ. Nevertheless, the setup may benefit from an autofocus or ‘focus lock’ module (home-built or commercially available) that may be added to the objective or sample stage in some embodiments.

Finally, some embodiments for an acquisition and processing scheme capable of combining the photoswitching techniques and the illumination/reflector setup of microscopy system 200 outlined above are described in greater detail below.
i. The sample is labeled with a reversibly switchable fluorescent marker such as rsEGP2.
ii. The sample is then activated with a standing wave of intermediate periodicity by adjusting G1 and G2 appropriately.
iii. The sample is imaged using the base optical microscope, e.g. the instant SIM.
iv. Steps ii) and iii) are repeated at two other phases of the standing wave, achieved by translating the piezoelectric actuator/mirror arrangement.
v. The sample is also activated with a standing wave of maximum periodicity (i.e. collimated incident and reflected beam at θ=0 degrees).
vi. The sample is then imaged using the base optical microscope, e.g. the instant SIM microscopy system.
vii. Steps v, vi are repeated for an additional phase of the standing wave, achieved by translating the piezoelectric actuator/mirror.
viii. Steps ii-vii are repeated as necessary at different focal planes in the sample, e.g. for acquiring a 3D imaging stack.
ix. Images are combined and deconvolved with Richardson-Lucy deconvolution to improve axial resolution.

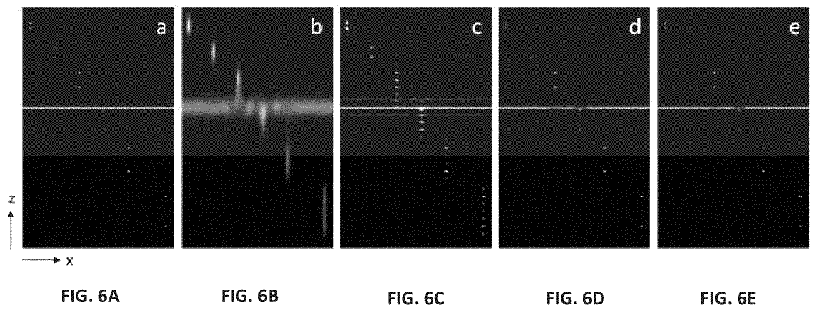

The resulting five images per focal plane were found to be sufficient for markedly increasing the axial resolution of the underlying microscope, as we have verified with simulations as illustrated in FIGS. 6A-6E. It was noted that rapid acquisition of all five images is necessary to prevent motion blur, and that building the illuminator/reflector on an instant SIM microscopy system ensures high speed image acquisition.

Simulations illustrating progressive improvement in axial resolution are reproduced in FIG. 6, beginning with FIG. 6A, a ‘perfect’ image of the object containing a series of features (line, dot pairs) spaced at various distances. FIG. 6B is an image of the object taken with an instant SIM without photoactivation or standing waves. FIG. 6C is an image of the object photoactivated with 150 nm periodicity standing wave (three phases) taken with an instant SIM system, and then deconvolved. Note that features are far better resolved, but artifacts (ringing) are evident, particularly for dot pairs spaced further apart. FIG. 6D is an image of the object photoactivated with a 300 nm periodicity standing wave (three phases), imaged with instant SIM microscopy system, and then deconvolved. Artifacts were shown to be reduced, but the dot pair with finest spacing is not resolved. FIG. 6E is an image of the object photoactivated with both 300 nm and 150 nm periodicity standing waves in sequence (five phases as described above). Note that features are well resolved without artifacts.

### Microscopy System for Achieving a Standing Wave Illumination Pattern Using Spatial Light Modulator

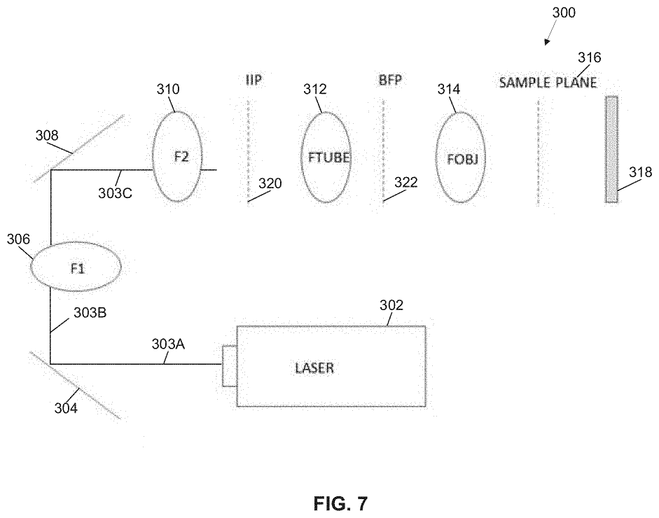

In a third embodiment of the microscopy system for utilizing the photoswitching and standing-wave illumination techniques, designated 300, is shown in FIG. 7. In some embodiments, the microscopy system 300 includes an optical layout similar to the second embodiment of the microscopy system 200 illustrated in FIG. 5, except that a spatial light modulator (SLM) 304 is positioned conjugate to the sample 316, and F1/F2 lenses 306 and 310 provide optional magnification in microscopy system 300. Additional optics may also be placed between F1/F2 lenses 306 and 310 to filter or condition the laser beam 303 prior to entry into the objective lens 314. In particular, as shown in FIG. 7, the microscopy system 300 may include a laser source 302 that emits a laser beam 303A which is reflected by the SLM 304 through a first F1 lens 306 and reflects off a translating reflective mirror 308 through a telescope composed of a second F2 lens 310 and a third FTUBE lens 312 onto the back focal plane of the objective lens (FOBJ) 314 before being finally focused onto the sample 316. A mirror 318 then reflects the illumination back onto the sample 316.

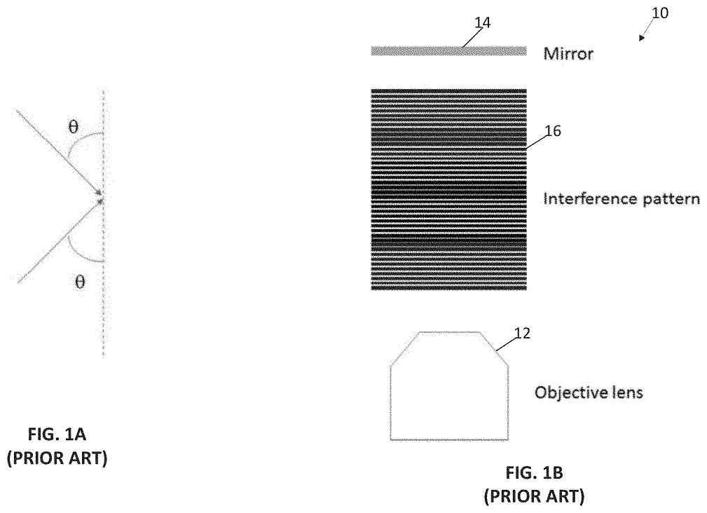

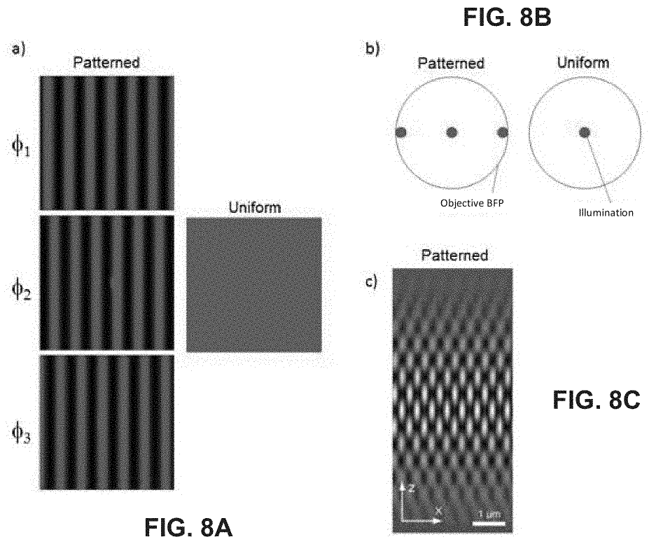

The SLM 304 provides an easy and flexible method for introducing both intermediate and finer (e.g. 300 nm, 150 nm patterns in FIG. 4) at the sample plane. By displaying sharp sinusoidal patterns at different phases on the SLM (FIG. 8A, left) and thus allowing 3-beam interference (FIG. 8B, left) at the sample, illumination with diffraction-limited axial modulation can be introduced and varied at the sample (FIG. 8C). By instead displaying a uniform pattern on the SLM (FIG. 8A, right; corresponding to on-axis illumination at the sample or a single centered illumination spot at the back focal plane, FIG. 8B, right), collimated, on-axis illumination is transmitted through the objective lens, which reflects at the mirror to produce sharply varying interference at the sample (e.g. as in FIG. 1B).

Various SLM patterns are shown in FIG. 8A as well as corresponding back focal plane (FIG. 8B) and sample (FIG. 8C) intensity patterns. By displaying sharp sinusoidal illumination at different phases shown in the three images of FIG. 8A, corresponding to 3 beam illumination illustrated in FIG. 8B (left) at the back focal plane 322 of the objective lens 314, sharp axial illumination is introduced at the sample (FIG. 8C). In contrast, if uniform illumination (FIG. 8A, FIG. 8B right) is used, uniform illumination is transmitted through the objective, resulting in a sharper illumination pattern akin to that in FIG. 1B after reflection from the mirror. In FIG. 8B, the blue circle represents the objective back focal plane and the red dots the illumination pattern at the back focal plane. In FIG. 8C, the illumination pattern is reproduced from Gustafsson, 2008.

The acquisition procedure performed by the microscopy system 300 will be very similar to the two-galvanometer setup:

Step 1: The sample 316 is labeled with a reversibly switchable fluorescent marker such as rsEGP2.

Step 2: the sample 316 is activated with a standing wave of intermediate periodicity by using the SLM 304 to display sharp sinusoidal illumination.

Step 3: The sample 316 is imaged using the base optical microscope arrangement, e.g. the instant SIM.

Step 4: Steps 2) and 3) are repeated at two other phases of the standing wave, achieved by displaying the appropriate patterns on the SLM 304.

Step 5: The sample 316 is activated with a standing wave of maximum periodicity (i.e. collimated incident and reflected beam at Θ=0 degrees), by changing to a uniform pattern on the SLM 304.

Step 6: The sample 316 is imaged using the base optical microscope, e.g. the instant SIM.

Step 7: Steps 5) and 6) are repeated for an additional phase of the standing wave, achieved by translating the piezoelectric actuator/mirror.

Step 8: Steps 2)-7) are repeated as necessary at different focal planes in the sample, e.g. for acquiring a 3D imaging stack.

Step 9: Images are combined and deconvolved with Richardson-Lucy deconvolution to improve axial resolution.

### Microscopy System for Generating a Sharp Axial Illumination Structure for Achieving Axial Super-Resolution

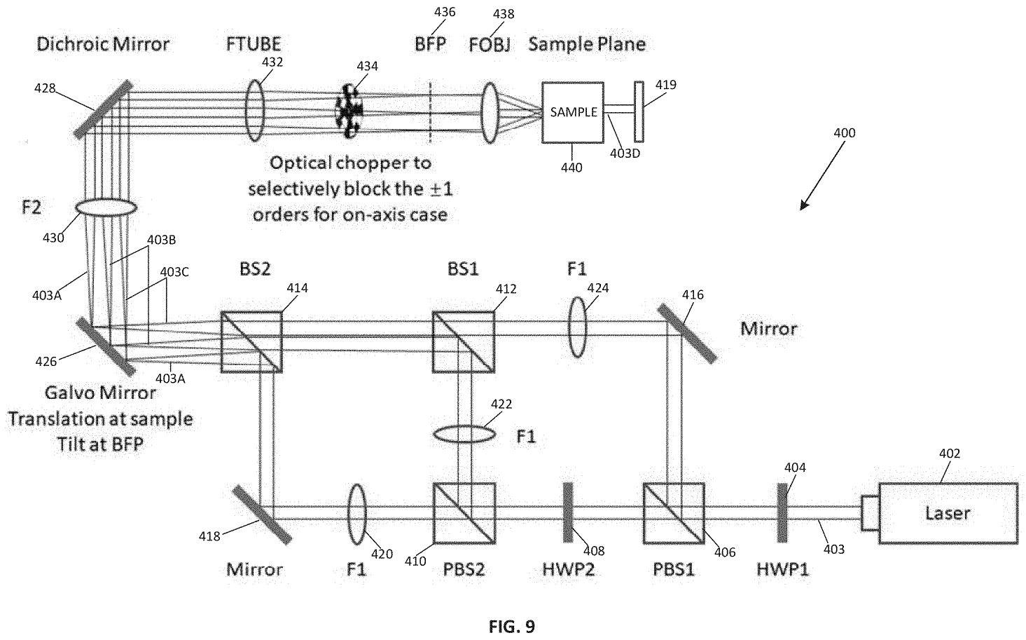

In a fourth embodiment of the microscopy system for generating a sharp axial illumination structure for achieving axial super-resolution, designated 400, is shown in FIG. 9. In this embodiment, a triple beam-splitting arrangement is used to generate three mutually coherent light beams split from a single light beam that interferes at the sample to produce axial fringes necessary for achieving higher axial resolution. In one aspect, a laser 402, for example a laser transmitting a single light beam 403 at a wavelength of 405 nm, is split into three split coherent light beams 403A, 403B, and 403C through a first beam splitter 406 and a second beam splitter 410, and then recombined through a first non-polarizing beam splitter 412 and second non-polarizing beam splitter 414. First, second, and third lenses 420, 422 and 424 having a focal length F1 are positioned prior to the first non-polarizing beam splitter 412 and second non-polarizing beam splitter 414 to ensure that the first, second, and third split light beams 403A, 403B, and 403C come into focus at a galvanometric mirror 426 positioned conjugate to the back focal plane 436 of an objective lens 438.

The polarization state of the first, second and third split light beams 403A, 403B, and 403C may be controlled using a first half wave plate 404 and second half wave plate 408. The first, second, and third split light beams 403A, 403B, and 403C at the back focal plane 436 provide illumination with a sharp axial structure as shown in FIG. 8C, while rotating the galvanometric mirror 426 changes the phase of the illumination structure as shown in FIG. 8A. As shown, a dichroic mirror 428 allows for integration with the other components of the microscopy system. In addition, an optical chopper 434 is positioned between the FTUBE lens 432 and the objective lens 438 and may be used to selectively block the outer two laser beams, e.g., first and third split light beams 403A and 403C, thereby allowing on-axis illumination by the second split light beam 403B. On-axis illumination by the second split-light beam 403B allows higher spatial frequency axial fringes after interference with the reflected light beam 403D from the mirror 419, which is located on the other side of the sample 440, opposite the objective lens 438 and coverslip that the sample 440 rests on. The resulting interference pattern produces a standing wave with maximum periodicity in the sample 440.

The acquisition procedure performed by the microscopy system 400 will be very similar to a two-galvanometer microscopy setup or a spatial light modulator (SLM) microscopy setup:

Step 1: The sample 440 is labeled with a reversibly switchable fluorescent marker, such as rsEGP2.

Step 2: The sample 440 is activated with a standing wave of intermediate periodicity by allowing the first, second and third light beams 403A, 403B, and 403C to propagate through the microscopy system 400, thereby enabling sinusoidal illumination at the sample 440.

Step 3: The sample 440 is imaged using a base optical microscope (not shown), such as an instant selective illumination microscopy.

Step 4: Steps 2 and 3 are repeated at four other phases of the standing wave which is achieved by rotating the galvanometer mirror 426 appropriately.

Step 5: The sample 440 is activated with a standing wave of maximum periodicity, for example collimated incident and reflected at Θ=0 degrees, using the optical chopper 436 to block the outer two laser beams, e.g., first and second light beams 403A and 403C.

Step 6: The sample 440 is imaged using the base optical microscope (not shown), such as an instant selective illumination microscopy.

Step 7: Steps 5 and 6 are repeated for an additional phase of the standing wave, thereby achieved by translating the mirror 419 by using a piezoelectric actuator (not shown).

Step 8: Steps 2 through 7 are repeated as necessary at different focal planes in the sample 440, for example by acquiring a three dimensional imaging stack.

Finally, the captured images are combined and deconvolved using a Richardson-Lucy deconvolution to improve axial resolution.

It was noted that since a nonlinear transition (photoswitching) is used in the microscopy systems 100, 200, 300 and 400 disclosed herein, in theory ‘unlimited’ resolution is possible by ‘saturating’ either ON or OFF states. Achieving saturation is simple in principle by turning up the 405 nm laser would be one way of saturating the ON state, leading to higher harmonics in each axial slice; however, the price that must be paid to read out this resolution improvement would be the acquisition of more raw images, but it is in principle possible given sufficiently photo-stable samples.

In one aspect, the techniques for photoswitching and standing wave illumination described herein may be applied to other microscopy systems to improve axial resolution. For example, the aforementioned techniques may be used with any type of widefield fluorescence or confocal microscopy systems to improve axial resolution.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

1. A method comprising:
a) labeling a sample with a reversibly switchable marker;
b) illuminating the sample with standing waves of an intermediate periodicity;
c) imaging the sample at a first instance;
d) repeating steps b) and c) at two other respective phases of the standing waves of intermediate periodicity;
e) imaging the sample at a second instance;
f) illuminating the sample with standing waves of a maximum periodicity;
g) imaging the sample in a third instance;
h) repeating steps f) and g) for an additional phase of the standing wave of maximum periodicity;
i) repeating steps b)-h) one or more times at different focal planes relative to the sample for acquiring a three-dimensional image of the sample.

2. The method of claim 1, wherein the reversibly switchable marker comprises a reversibly switchable fluorescent maker.

3. The method of claim 1, wherein the reversibly switchable marker comprises an rsEGP2 marker.

4. The method of claim 1, wherein the sample is imaged using instant structured illumination microscopy.

5. The method of claim 1, wherein repeating steps b)-h) comprises repeating steps b)-h) five times to obtain five images per focal plane.

6. The method of claim 5, further comprising:
combining and devolving each of the images using Richardson-Lucy or other deconvolution process to produce a composite image.

7. The method of claim 1, wherein illuminating the sample comprises varying an angle of a standing wave pattern relative to a plane of the sample for changing a periodicity of the phase of each standing wave.

8. A microscopy system comprising:
an illumination source for producing a collimated light beam;
an objective lens for receiving the collimated light beam; and
a mirror in operative association with the objective lens for reflecting the collimated light beam such that a reflected collimated light beam is produced that results in a standing wave pattern illuminating a sample.

9. The microscopy system of claim 8, further comprising:
a coverslip that defines a surface in which the sample is located.

10. The microscopy system of claim 8, wherein interference between the collimated light beam and the reflected collimated light beam produces the standing wave pattern.

11. The microscopy system of claim 8, further comprising:
a piezoelectric device coupled to the mirror for translating the mirror (106) relative to the sample.

12. The microscopy system of claim 8, further comprising:
an imaging system for acquiring five images of the sample per focal plane.

13. The microscopy system of claim 8, further comprising:
at least one reversibly switchable fluorescent molecule in association with the sample for producing a fluorescent excitation in the sample.

14. A microscopy system comprising:
an illumination source for transmitting a light beam;
a first scanning mirror positioned in a location conjugate to a sample for reflecting the transmitted light beam onto a second scanning mirror that reflects the reflected transmitted light beam through an objective lens for illuminating the sample that generates an illumination from the sample; and
a mirror for reflecting the illumination from the sample back onto the sample for generating a standing wave pattern.

15. The microscopy system of claim 14, wherein a first lens is in 4f configuration with a second lens.

16. The microscopy system of claim 14, wherein the first scanning mirror tilts the standing wave pattern at a sample plane of the sample for changing a standing wave periodicity of the standing wave pattern.

17. The microscopy system of claim 14, wherein the mirror is translated along an axis relative to the sample.

18. The microscopy system of claim 14, further comprising:
an intermediate lens arrangement in operative association with the objective lens for ensuring that the second scanning mirror is conjugate to a back focal plane of the objective lens.

19. The microscopy system of claim 14, further comprising:
an imaging system for capturing five images of the sample per focal plane.

20. The microscopy system of claim 14, wherein the mirror comprises a dichroic mirror.

21. A method comprising:
a) labeling a sample with a reversibly switchable fluorescent marker;
b) illuminating the sample with standing waves of an intermediate periodicity by using a spatial light modulator to display a sharp sinusoidal illumination;
c) imaging the sample at a first instance;
d) repeating steps b) and c) at two other respective phases of the standing waves of the intermediate periodicity achieved by displaying appropriate patterns on the spatial light modulator;
e) imaging the sample at a second instance;
f) illuminating the sample with standing waves of a maximum periodicity by changing to a uniform pattern on the spatial light modulator;
g) imaging the sample in a third instance;
h) repeating steps f) and g) for an additional phase of the standing waves of maximum periodicity achieved by translating a piezoelectric actuator/mirror;
i) repeating steps b)-h) one or more times at different focal planes relative to the sample for acquiring a three-dimensional image of the sample.

22. The method of claim 21, wherein the reversibly switchable fluorescent marker comprises a reversibly switchable fluorescent maker.

23. The method of claim 21, wherein the reversibly switchable fluorescent marker comprises an rsEGP2 marker.

24. The method of claim 21, wherein the sample is imaged using instant structured illumination microscopy.

25. The method of claim 21, wherein repeating steps b)-h) comprises repeating steps b)-h) five times to obtain five images per focal plane.

26. The method of claim 25, further comprising:
combining and devolving each of the images using Richardson-Lucy (or other) deconvolution process to produce a composite image.

27. The method of claim 1, wherein illuminating the sample comprises varying an angle of a standing wave pattern relative to a plane of the sample for changing a periodicity of the phase of each standing wave.

28. A microscopy system comprising:
an illumination source for producing a collimated light beam;
an objective lens for receiving the collimated light beam; and
a spatial light modulator in operative association with the objective lens for reflecting the collimated light beam such that a reflected collimated light beam is produced that results in a standing wave pattern illuminating a sample.

29. The microscopy system of claim 28, further comprising:
a coverslip that defines a surface in which the sample is located.

30. The microscopy system of claim 28, wherein interference between the collimated light beam and the reflected collimated light beam produces the standing wave pattern.

31. The microscopy system of claim 28, further comprising:
a piezoelectric device coupled to a mirror for translating the mirror relative to the sample.

32. The microscopy system of claim 28, further comprising:
an imaging system for acquiring five images of the sample per focal plane.

33. The microscopy system of claim 28, further comprising:
at least one reversibly switchable fluorescent molecule in association with the sample (316) for producing a fluorescent excitation in the sample.

34. A method comprising:
a) labeling a sample with a reversibly switchable marker;
b) activating the sample with standing waves of an intermediate periodicity by allowing first, second and third laser beams to enable display of a sharp sinusoidal illumination at the sample;
c) imaging the sample at a first instance;
d) repeating steps b) and c) at four other respective phases of the standing waves of intermediate periodicity achieved by rotating a galvanometer mirror;
e) activating the sample with a standing wave of maximum periodicity using an optical chopper;
f) imaging the sample in a second instance;
g) repeating steps e) and f) for an additional phase of the standing wave; and
h) repeating steps a) through g) at different planes of the sample for acquiring a three-dimensional image of the sample.

35. The method of claim 34, wherein the reversibly switchable marker comprises a reversibly switchable fluorescent maker.

36. The method of claim 34, wherein the reversibly switchable marker comprises an rsEGP2 marker.

37. The method of claim 34, wherein the sample 440 is imaged using instant structured illumination microscopy.

38. (canceled)

39. The method of claim 34, wherein illuminating the sample comprises varying an angle of a standing wave pattern relative to a plane of the sample for changing a periodicity of the phase of each standing wave.

40. A microscopy system comprising:
an illumination source for producing a light beam 4;
first polarizing beam splitter and a second polarizing beam splitter in operative communication with the illumination source for splitting the light beam into first split light beam, second split light beam, and third split light beam;
first non-polarizing beam splitter and second non-polarizing beam splitter for recombining first split light beam, second split light beam, and third split light beam;
a galvanometer mirror in operative communication with the first and second non polarizing beam splitters;
an objective lens for receiving the first light beam, second light beam, and third light beam; and
an optical chopper in operative association with the objective lens for reflecting the light beam such that a reflected collimated light beam is produced that results in a standing wave pattern illuminating a sample.

41. The microscopy system of claim 40, wherein the first, second, and third split light beams comprises mutually coherent light beams that interfere at the sample to produce lower spatial frequency axial fringes for producing higher axial resolution of the sample (440).
